(19) United States
(12) Patent Application Publication
(10) Pub. No.: US 2013/0261675 A1
(43) Pub. Date: Oct. 3, 2013

(54) LOOSE THREAD FORM FOR VARIABLE ANGLE LOCKING SYSTEMS

(71) Applicant: Daniel Duane Fritzinger, Warsaw, IN (US)

(72) Inventor: Daniel Duane Fritzinger, Warsaw, IN (US)

(21) Appl. No.: 13/432,392

(22) Filed: Mar. 28, 2012

(51) Int. Cl. A61B 17/86; A61B 17/80

(52) U.S. Cl. 606/291; 606/308

(57) ABSTRACT

A variable angle locking screw comprising a head portion having helical threads and an elongated threaded shaft portion extending from the head portion, wherein a gap between the helical threads is substantially greater than the thickness of the threads

### TECHNICAL FIELD

The present invention generally relates to thread form designs for fasteners, and more particularly to loose thread forms for variable angle locking systems.

### BACKGROUND OF THE INVENTION

The statements in this section merely provide background information related to the present disclosure and should not be construed as constituting prior art.

The use of orthopedic fastening devices, such as bone screws, has greatly aided the medical field in the treatment of bone fractures. More particularly, when treating bone fractures, it is sometimes generally necessary to surgically reposition fragmented bone members in various anatomically acceptable orientations. To fasten the repositioned bone members together in order to facilitate the healing process, bone screws are often used as part of the stabilization process (e.g., either by fastening two or more bone members together, or by securing an orthopedic appliance or bone plate to the bone's surface). Sometimes it is beneficial to orient the bone screw at an angle that is non-perpendicular to the orthopedic appliance during the stabilization process—for instance, to avoid poor bone stock or fracture lines. Many variable angle locking systems, however, are difficult to manipulate, particularly in terms of variably orienting the screw relative to the fixture. In addition, such locking systems also do not sufficiently prohibit relative motion between the screw and the orthopedic fixture to which it is secured.

The present invention is intended to improve upon and resolve some of these known deficiencies within the relevant art.

### SUMMARY OF THE INVENTION

In accordance with one aspect of the present invention, a variable angle locking screw is provided and comprises a head portion having helical threads and an elongated threaded shaft portion extending from the head portion. In accordance with this embodiment, a gap between the helical threads is substantially greater than the thickness of the threads.

In accordance with yet another aspect of the present invention, a variable angle locking screw assembly is provided and comprises a plate material having an opening defining a first axis therethrough, and a screw including a head portion having helical threads and an elongated threaded shaft portion extending from the head portion. The shaft portion is insertable into the opening at more than one angle relative to the first axis to lock the screw to the plate material, and a gap between the helical threads is substantially greater than the thickness of the threads.

In still another aspect of the present invention, a variable angle locking screw assembly is provided and comprises a screw including a head portion having helical threads and an elongated threaded shaft portion extending from the head portion, and a plate material having an opening defining a first axis therethrough, the opening having threads and being configured to lockably receive, at more than one angle relative to the first axis, the shaft portion as it inserted therethrough. In accordance with this embodiment, a gap between the helical threads is up to about two times greater than the thickness of the threads, and the helical threads are configured to mate with threads of the opening as the screw is locked to the plate material.

Still other objects and benefits of the invention will become apparent from the following written description along with the accompanying figures.

### BRIEF DESCRIPTION OF THE DRAWINGS

The above-mentioned aspects of the present invention and the manner of obtaining them will become more apparent and the invention itself will be better understood by reference to the following description of the embodiments of the invention taken in conjunction with the accompanying drawings, wherein:

Corresponding reference characters indicate corresponding parts throughout the several views. Although the exemplification set out herein illustrates embodiments of the invention, in several forms, the embodiments disclosed below are not intended to be exhaustive or to be construed as limiting the scope of the invention to the precise forms disclosed.

FIG. 1 is a profile view of an illustrative variable angle locking bone screw in accordance with the teachings of the present invention;

FIG. 2 is an elevated perspective view of the illustrative variable angle locking bone screw of FIG. 1;

FIG. 3 is a profile view of an illustrative variable angle locking bone screw head having a loose thread form in accordance with the teachings of the present invention;

FIG. 4 is a cross-section of an illustrative bone plate having a threaded hole in accordance with the teachings of the present invention;

FIG. 5 is a cross-section of an illustrative variable angle locking bone screw having an on-axis assembly to a threaded bone plate in accordance with the teachings of the present invention;

FIG. 6 is a cross-section of an illustrative variable angle locking bone screw having an off-axis assembly to a threaded bone plate in accordance with the teachings of the present invention;

FIG. 7 is a top view of an illustrative bone plate having a variable angle locking bone screw threaded therethrough in an off-axis orientation in accordance with the teachings of the present invention; and

FIG. 8 is a cross-section of the illustrative variable angle locking bone screw threaded through the bone plate of FIG. 7 taken along line 8-8.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

Unless defined otherwise, all technical and scientific terms used herein have the same meaning as commonly understood by one of ordinary skill in the art to which this invention belongs. Although any method and materials similar or equivalent to those described herein can be used in the practice or testing of the present invention, the specific methods and materials are now described. Moreover, the techniques employed or contemplated herein are standard methodologies well known to one of ordinary skill in the art and the materials, methods and examples are illustrative only and not intended to be limiting.

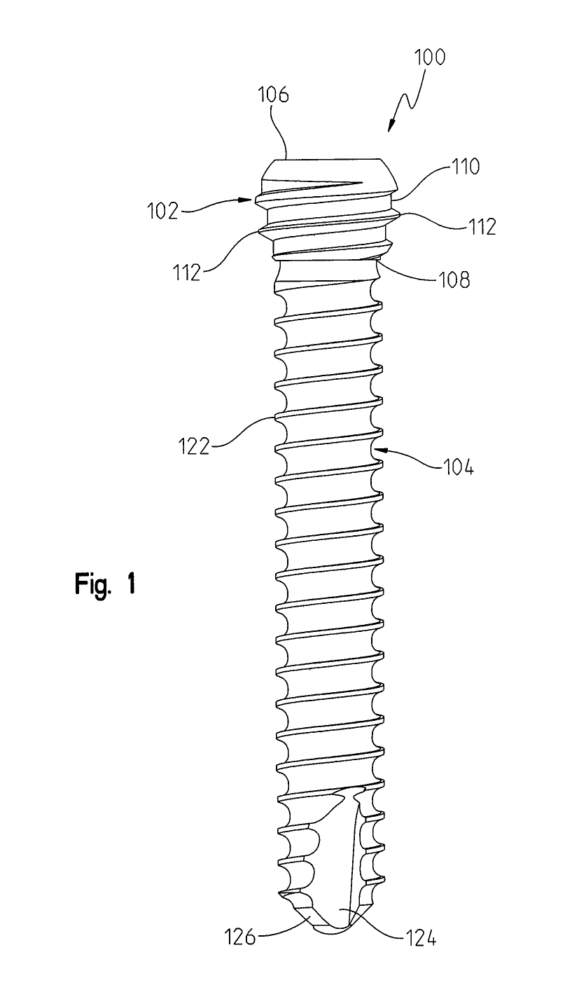

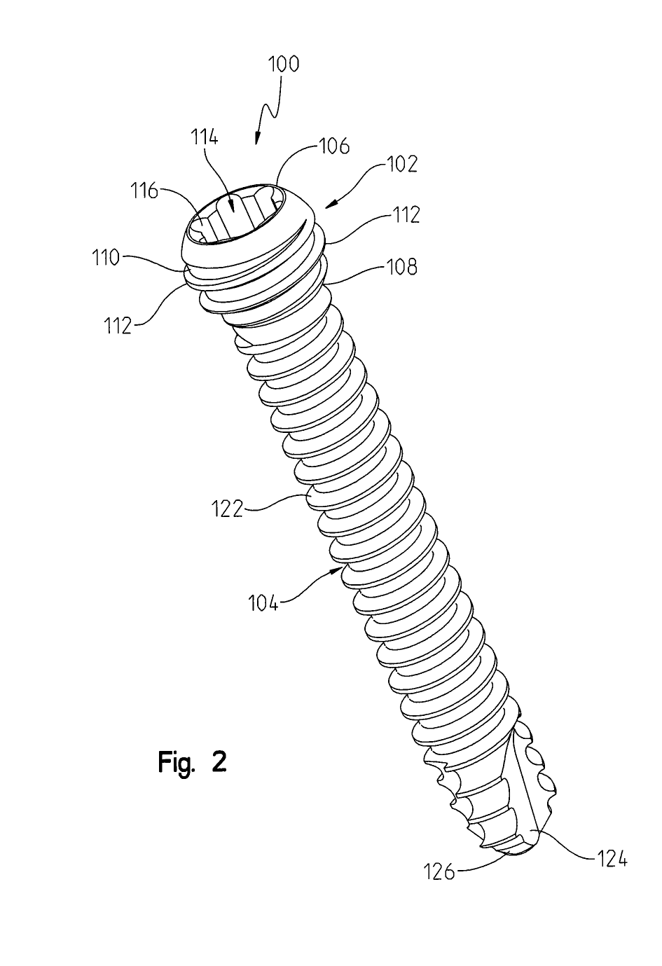

Referencing FIGS. 1 and 2, an illustrative variable angle locking screw 100 in accordance with the teachings of the present invention is shown. The screw 100 includes a head 102 and an elongated shaft 104 extending from the head. In accordance with certain aspects of the present invention, the head 102 includes a substantially planar top surface 106 spaced apart from a conical bottom surface 108 that tapers and narrows in diameter as the distance from the top surface 106 increases. A circumferential surface 110 of the head 102, which extends between the top surface 106 and the bottom surface 108, is substantially smooth but for helical threads 112 that extend around the circumference of the head. This circumferential surface 110 in combination with the threads 112 delineates the horizontal widthwise dimension of the screw head 102. More specifically, the horizontal widthwise dimension of the screw head 102 is larger than the horizontal widthwise dimension of the elongated shaft 104.

In accordance with certain illustrative aspects of the present invention, an opening 114 extends from the top surface 106 normally into the interior of the head 102 and is bounded by a series of walls 116 that intersect a conically depressed floor (not shown). The conical floor extends partially into the interior of the elongated shaft 104, which extends normally from the bottom portion 108 of the head 102. The elongated shaft 104 is generally cylindrical in horizontal cross-section and includes helical threads 122 distributed about its circumference from proximate the bottom portion 108 of the head 102 to the tip 124 of the elongated shaft 104, which includes conical portion 126 transitioning from the generally circular cross-section of the elongated shaft 104 to the tip 124. It should be noted that the horizontal widthwise dimension of the head 102 is substantially larger than the widthwise dimension of the elongated shaft 104 so that the bottom surface 108 of the head that extends laterally outward (i.e., widthwise) beyond the elongated shaft 104 provides a conical plateau.

As should be understood and appreciated herein, the locking screw 100 of the present invention is intended to be utilized with various variable angle locking assemblies. In accordance with one illustrative embodiment, the locking screw 100 is adapted to be received by one or more through holes of an orthopedic appliance or plate material (e.g., a bone plate) to mount the appliance to bodily tissue such as, without limitation, bone. Because the orthopedic appliance may not always be planar, there may be instances where the surgical screw 100 is oriented at an angle other than perpendicular with respect to a vertical axis of the through hole (or with respect to the bottom and top surfaces of the orthopedic appliance). In addition, it may be desirable to angle the screw 100 away from areas of poor bone stock or fracture lines.

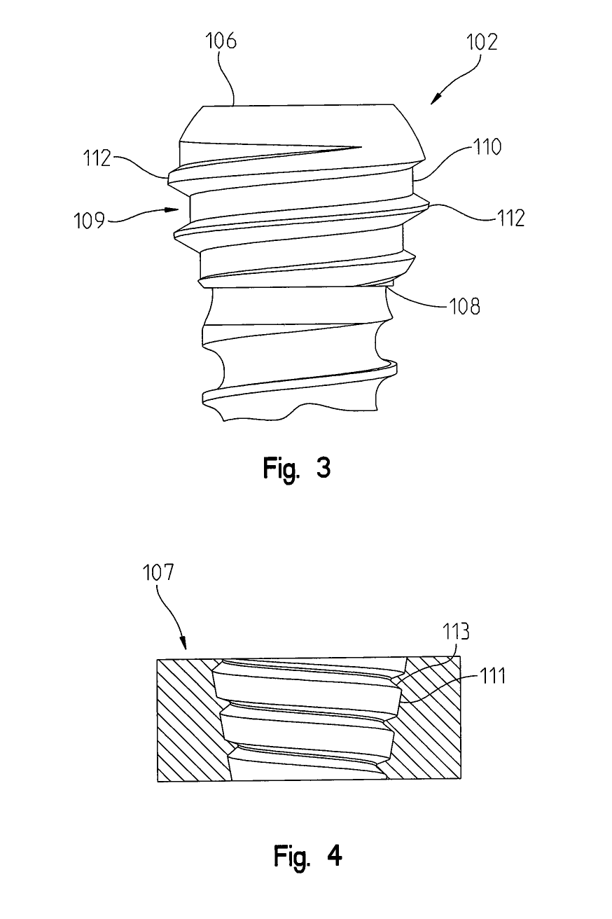

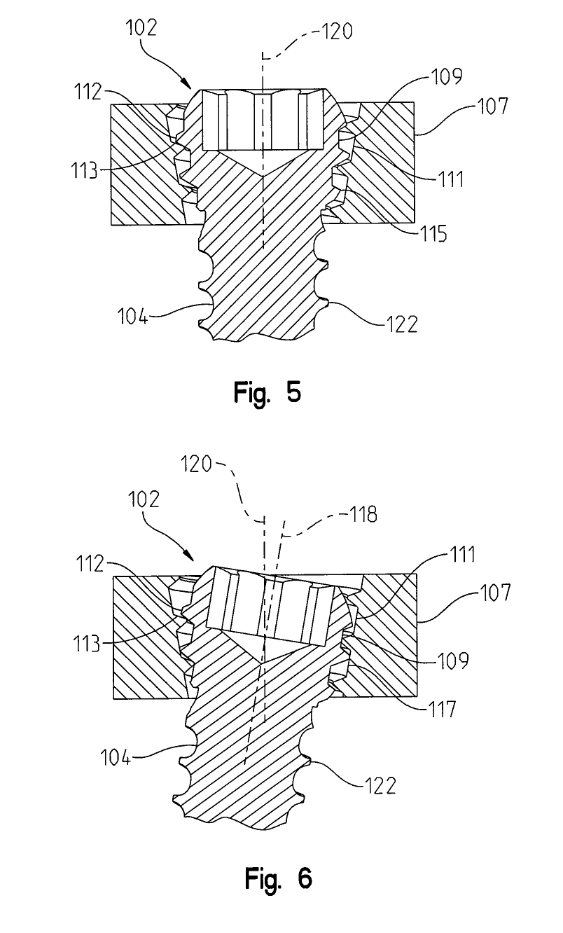

With particular reference to FIG. 3, the locking screw 100 helical threads 112 extend around the circumference of the head 102. Depending on the use and application of the locking screw 100, it should be understood and appreciated that the helical threads 112 may be spherical, conical or spherical-conical in thread form. In accordance with these embodiments, however, it is particularly beneficial if the gap or space 109 between the helical threads 112 is substantially greater than the thickness of the threads. Alternatively, as shown in FIG. 4, which depicts a cross-sectional view of a bone plate 107 having a threaded hole, it should be understood and appreciated herein that the inventive design may instead be configured such that the gap or space 111 between the threads 113 of the threaded hole of the bone plate 107 is substantially greater than the thickness of the threads. Because of the inventive configuration of the gap-to-thread thickness ratio and spacing disclosed herein, the screw 100 can be installed at various angles with respect to the bone plate 107 and still achieve a lockable fixation thereto. More particularly, when the screw 100 is initially assembled into the plate 107, the threads 112 of the screw head 102 will mate with the threads 113 of the plate 107, thereby causing the screw 100 to thread into the plate. As the screw 100 is tightened, the threads 112, 113 will eventually wedge against each other to lock the screw head 102 to the plate 107. FIG. 5, for instance, shows an on-axis assembly point when the threads 112, 113 begin to lock to each other (the point represented by the reference numeral 115).

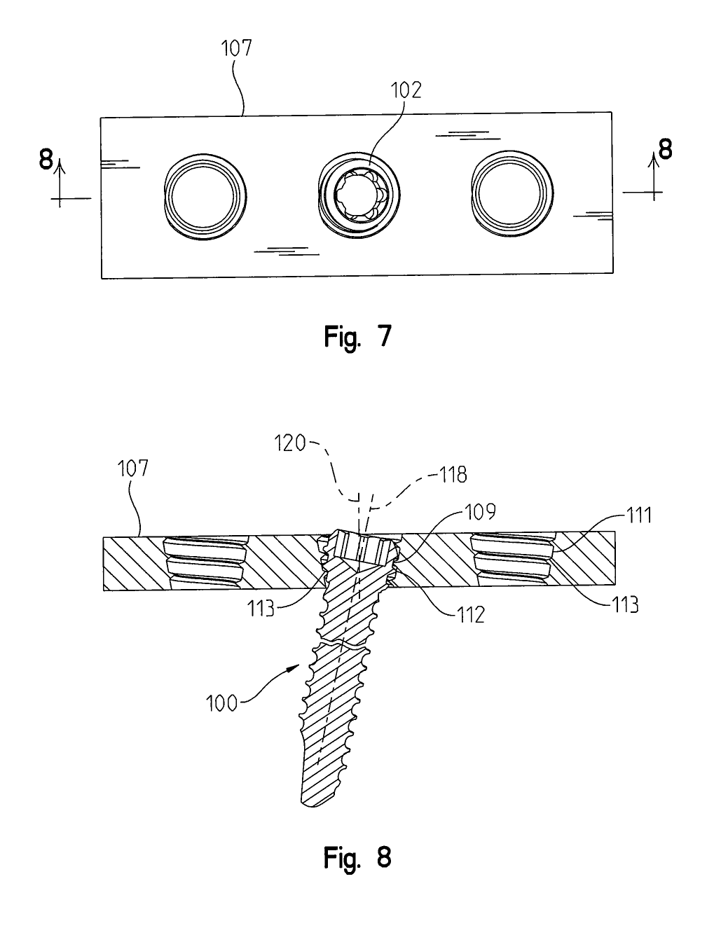

As shown in FIG. 6, due to the enlarged gap 109 between the threads 112, the screw 100 can be inserted off-axis (i.e., the axis of the screw is represented by reference numeral 118 and the axis of the plate is represented by reference numeral 120) and the threads will still lockably engage each other. In other words, even when positioned off-axis, as the screw is tightened, the threads 112, 113 will eventually wedge against each other to lock the screw head 102 to the plate 107 (see point represented by the reference numeral 117). It should be understood and appreciated herein that those of skill in the art will be able to adjust the ratio of the gap-to-thread thickness of either the screw head and/or the bone plate to determine the maximum amount of off-axis screw angle. In accordance with certain specific embodiments, for instance, the ratio of the gap-to-thread thickness can be up to about 2:1. In accordance with one illustrative embodiment depicted in FIGS. 7-8, the gap 109 between the threads 112 of the screw 100 is about two times greater than the thickness of the threads. Such an orientation makes it possible to achieve a maximum off-axis angular alignment 118 of about 12 degrees in any direction from a central on-axis alignment point 120 of the plate 107 (i.e., creates a 24 degree cone of angulation). To achieve this variable off-axis angular orientation with respect to the plate 107, those of skill in the art will recognize that the gap 109 between the threads 112 of the screw head 102 should be about twice the size of the threads 113 of the bone plate 107. While this illustrative example demonstrates a 12 degree off-axis angular alignment, it should be understood that other angular dimensions can be achieved by those of skill in the art and still fall within the scope and spirit of the present invention.

While those of skill in the art will understand and appreciate that the dimensional characteristics of the inventive variable angle locking loose thread design disclosed herein can vary depending on the intended use and application of the screw, in accordance with one illustrative embodiment, the female portion of the loose thread design of the bone screw can have the following approximate dimensions: a thread thickness at the thread root of about 0.013″ and an approximate pitch of about 0.035″. Similarly, the male portion of the loose thread design of the bone screw can have the following approximate dimensions: a thread thickness at the thread root of about 0.011″, an approximate pitch of about 0.015″. In accordance with this specific illustration, the approximate tolerances can be about +/−0.002″.

The aforementioned may, in exemplary forms thereof, be manufactured from titanium or stainless steel. However, it should be understood and appreciated herein that any suitable material may be utilized to fabricate the aforementioned components, including, without limitation, plastics, ceramics, metals, and alloys of the foregoing.

While an exemplary embodiment incorporating the principles of the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

The terminology used herein is for the purpose of describing particular illustrative embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations).

1. A variable angle locking screw comprising:
a head portion having helical threads; and
an elongated threaded shaft portion extending from the head portion;
wherein a gap between the helical threads is substantially greater than the thickness of the threads.

2. The variable angle locking screw of claim 1, wherein the gap between the helical threads is about two times greater than the thickness of the threads.

3. The variable angle locking screw of claim 1, wherein the helical threads have a spherical, conical or spherical-conical thread form.

4. The variable angle locking screw of claim 1, wherein the helical threads are configured to lockably mate with threads of a plate material at more than one angle relative to a first axis as the screw is advanced through an opening of the plate material.

5. The variable angle locking screw of claim 4, wherein the plate material is a bone plate.

6. The variable angle locking screw of claim 4, wherein the first axis is arranged in substantially perpendicular correspondence to a first plane of the plate material.

7. The variable angle locking screw of claim 6, wherein the helical threads are configured to lockably mate with the threads of the plate material such that the shaft portion is positioned at an off-axis angle of up to about 12 degrees in any direction relative to the first axis.

8. The variable angle locking screw of claim 1, further comprising an opening extending normally from a top surface of the head portion and into an interior portion that is bounded by a series of walls intersecting a conically depressed floor.

9. A variable angle locking screw assembly comprising:
a plate material having an opening defining a first axis therethrough; and
a screw including a head portion having helical threads and an elongated threaded shaft portion extending from the head portion, the shaft portion being insertable into the opening at more than one angle relative to the first axis to lock the screw to the plate material;
wherein a gap between the helical threads is substantially greater than the thickness of the threads.

10. The variable angle locking screw assembly of claim 9, wherein the gap between the helical threads is up to about two times greater than the thickness of the threads.

11. The variable angle locking screw assembly of claim 9, wherein the helical threads have a spherical, conical or spherical-conical thread form.

12. The variable angle locking screw assembly of claim 9, wherein the plate material is a bone plate.

13. The variable angle locking screw assembly of claim 9, wherein the first axis is arranged in substantially perpendicular correspondence to a first plane of the plate material.

14. The variable angle locking screw assembly of claim 13, wherein the helical threads are configured to lockably mate with threads of the plate material such that the shaft portion is positioned at an off-axis angle of up to about 12 degrees in any direction relative to the first axis.

15. The variable angle locking screw assembly of claim 9, further comprising an opening extending normally from a top surface of the head portion and into an interior portion that is bounded by a series of walls intersecting a conically depressed floor.

16. A variable angle locking screw assembly comprising:
a screw including a head portion having helical threads and an elongated threaded shaft portion extending from the head portion; and
a plate material having an opening defining a first axis therethrough, the opening having threads and being configured to lockably receive, at more than one angle relative to the first axis, the shaft portion as it inserted therethrough;
wherein a gap between the helical threads is up to about two times greater than the thickness of the threads; and
wherein the helical threads are configured to mate with threads of the opening as the screw is locked to the plate material.

17. The variable angle locking screw assembly of claim 16, wherein the plate material is a bone plate.

18. The variable angle locking screw assembly of claim 16, wherein the helical threads have a spherical, conical or spherical-conical thread form.

19. The variable angle locking screw assembly of claim 16, wherein the first axis is arranged in substantially perpendicular correspondence to a first plane of the plate material and wherein the helical threads are configured to lockably mate with the threads of the plate material such that the shaft portion is positioned at an off-axis angle of up to about 12 degrees in any direction relative to the first axis.

20. The variable angle locking screw assembly of claim 16, further comprising an opening extending normally from a top surface of the head portion and into an interior portion that is bounded by a series of walls intersecting a conically depressed floor.
